SEQUENCE LISTING (10 sequences)

SEQ ID NO: 1 - DNA, 36 bp, Escherichia coli
aagaggagctcattacgtgactgattctaacaaaac

SEQ ID NO: 2 - DNA, 47 bp, Escherichia coli
catcgtcgaaacggtatttgtcatatattccaccagctatttgttag

SEQ ID NO: 3 - DNA, 36 bp, Escherichia coli
aagagtctagaattacgtgactgattctaacaaaac

SEQ ID NO: 4 - DNA, 47 bp, Escherichia coli
cattacggaatcgatatcacgcatatattccaccagctatttgttag

SEQ ID NO: 5 - DNA, 47 bp, Zymomonas mobilis
ctaacaaatagctggtggaatatatgacaaataccgtttcgacgatg

SEQ ID NO: 6 - DNA, 33 bp, Zymomonas mobilis
aagagtctagattagataccggcacctgcatat

SEQ ID NO: 7 - DNA, 47 bp, Zymomonas mobilis
ctaacaaatagctggtggaatatatgcgtgatatcgattccgtaatg

SEQ ID NO: 8 - DNA, 35 bp, Zymomonas mobilis
aagaggtatatattattaggcaacagcagcgcgct

SEQ ID NO: 9 - DNA, 44 bp, Zymomonas mobilis
gttgcctaaaagaggtatatattaatgaccgaagccgaatggtg

SEQ ID NO: 10 - DNA, 33 bp, Zymomonas mobilis
aagaggtcgacttatttgctccagtacacgtcg

(19) United States
(12) Patent Application Publication
(10) Pub. No.: US 2017/0268023 A1
(43) Pub. Date: Sep. 21, 2017

(54) METHOD OF PRODUCING ALCOHOL

(71) Applicant: Toray Industries, Inc., Tokyo (JP)

(72) Inventors: Kaoru Amagai, Kamakura (JP); Kenji Sawai, Tokyo (JP); Ryota Aiba, Otsu (JP); Katsushige Yamada, Kamakura (JP)

(73) Assignee: Toray Industries, Inc., Tokyo (JP)

(21) Appl. No.: 15/531,789

(22) PCT Filed: Dec. 10, 2015
(86) PCT No.: PCT/JP2015/084599

§ 371 Date: May 31, 2017

(30) Foreign Application Priority Data
Dec. 11, 2014 (JP) 2014-250705

(51) Int. Cl. C12P 7/06; C12N 15/63; C12Q 1/68

(52) U.S. Cl. CPC C12P 7/065; C12Q 1/686; C12N 15/63

(57) ABSTRACT

A method of producing alcohol includes culturing a microorganism capable of producing alcohol as a major product from a material containing pentose, wherein the microorganism is a microorganism having an enhanced Entner-Doudoroff (ED) pathway, and alcohol yield from xylose or arabinose based on sugar consumption is improved compared to alcohol yield based on sugar consumption before enhancement of the ED pathway.

### TECHNICAL FIELD

This disclosure relates to a method of producing alcohol from a material containing pentose using a microorganism having an enhanced Entner-Doudoroff (ED) pathway.

### BACKGROUND

Conventionally, alcohols and organic acids have been industrially produced by fermentation methods using various microorganism strains. Usually, as carbon sources, carbohydrates such as hexose (6-carbon sugar), pentose (5-carbon sugar), and triose, organic acids, and alcohols, are used. Examples of the hexose include glucose, fructose, mannose, sorbose, and galactose. Examples of the pentose include arabinose, xylose, and ribose. However, the above carbohydrates, and other conventionally used carbon sources currently industrially used are rather expensive. Therefore, less expensive alternative sources for production of alcohol have been demanded.

Since cellulosic biomass is easily available and less expensive than carbohydrates, maize, sugar cane, and other carbon sources, it is a preferred material for production of alcohol and organic acids. Normal amounts of cellulose, hemicellulose, and lignin in biomass are as follows. Biomass usually contains about 40 to 60% cellulose, 20 to 40% hemicellulose, 10 to 25% lignin, and 10% other components. The cellulose fraction is constituted of polymers of hexose, which is usually glucose. The hemicellulose fraction is mainly constituted of pentose including xylose and arabinose. For conversion of the above carbohydrates to useful compounds such as alcohols and organic acids, the efficiency of conversion of cellulosic biomass, which is a fermentation material that can be used, needs to be increased.

Examples of major microorganisms used for alcohol production include yeast belonging to the genus Saccharomyces and bacteria belonging to the genus Zymomonas. Usually, those microorganisms efficiently produce alcohol from sugars such as glucose, but cannot produce alcohol from pentoses such as xylose and arabinose. Thus, to improve the yield in production of alcohol using biomass as a material, an enzyme or a gene involved in the metabolism of pentose needs to be introduced to a microorganism used for alcohol production, to construct a transformed microorganism capable of producing alcohol using pentose as a substrate. U.S. Pat. No. 5,843,760 A discloses construction of a microorganism capable of producing ethanol from xylose or arabinose, which construction was carried out by transformation of a bacterium belonging to the genus Zymomonas with a gene involved in the metabolism of xylose or arabinose. JP 2012-115248 A discloses yeast that shows an improved fermentation yield of ethanol using xylose as a sugar source, which yeast was obtained by introduction of genes involved in the xylose metabolism into yeast belonging to the genus Saccharomyces.

Many microorganisms such as intestinal bacteria have the Entner-Doudoroff (ED) pathway as a glucose metabolic pathway. The pathway is composed of 6-phosphogluconate dehydratase (EDD) and 2-keto-3-deoxy-6-phosphogluconate aldolase (EDA), which catalyze the reaction from 6-phosphogluconate to 2-keto-3-deoxy-6-phosphogluconate.

US 2011/0165660 A describes an attempt to improve ethanol fermentation through the Entner-Doudoroff (ED) pathway by introduction of genes involved in the Entner-Doudoroff (ED) pathway into yeast belonging to the genus Saccharomyces to reinforce its glucose metabolic pathway. Similarly, US 2010/0120105 A discloses an attempt to improve the efficiency of production of isobutanol from glucose, by introduction of genes involved in the Entner-Doudoroff (ED) pathway into Escherichia coli.

As described above, the major sugar components obtained by hydrolysis of cellulosic biomass is a mixture containing glucose as a hexose, as well as xylose and arabinose as pentoses. Effective use of pentose has therefore been studied by, for example, a method in which an improved alcohol fermentation microorganism is used for production of alcohol by microbial fermentation from a material containing pentose. However, the alcohol productivity is still low when alcohol fermentation is carried out using pentose as a material, which is problematic.

The Applicant hereby incorporates by reference the sequence listing contained in the ASCII text file titled SequenceListing.txt, created May 23, 2017 and having 1.70 KB of data.

### SUMMARY

We discovered that, by use of a microorganism having an enhanced Entner-Doudoroff (ED) pathway, alcohol productivity from a material containing pentose can be improved.

We thus provide (1) to (7):

(1) A method of producing alcohol, comprising culturing a microorganism capable of producing alcohol as a major product from a material containing pentose, wherein the microorganism is a microorganism having an enhanced Entner-Doudoroff (ED) pathway, and the alcohol yield based on sugar consumption is improved compared to the alcohol yield based on sugar consumption before the enhancement of the ED pathway.
(2) The method of producing alcohol according to (1), wherein the alcohol yield based on sugar consumption in the microorganism is improved by not less than 5% compared to the alcohol yield based on sugar consumption before the enhancement of the ED pathway.
(3) The method of producing alcohol according to (1) or (2), wherein the microorganism is a microorganism showing enhanced expression of a gene(s) encoding at least one selected from the group consisting of glucose-6-phosphate dehydrogenase, 6-phosphogluconolactonase, phosphogluconate dehydratase, and 2-dehydro-3-deoxy-6-phosphogluconate aldolase.
(4) The method of producing alcohol according to any one of (1) to (3), wherein the alcohol is ethanol.
(5) The method of producing alcohol according to any one of (1) to (4), wherein the microorganism is a microorganism belonging to the genus Escherichia.
(6) The method of producing alcohol according to any one of (1) to (5), wherein the microorganism is Escherichia coli.
(7) The method of producing alcohol according to any one of (1) to (6), wherein the pentose is xylose or arabinose.

Enhancement of the Entner-Doudoroff (ED) pathway of a microorganism enables efficient alcohol fermentation from a material containing pentose. This leads to achievement of an efficient alcohol production method using as materials xylose and arabinose contained in sugar components obtained by hydrolysis of cellulosic biomass, that does not compete with food and is easily available, rather than using materials mainly containing glucose (cellulose and starch), whose competition with food is unavoidable. Thus, a significant social effect can be expected.

### BRIEF DESCRIPTION OF THE DRAWINGS

FIG. 1 is a schematic diagram showing the reaction pathway in the activity measurement of the Entner-Doudoroff (ED) pathway.

### DETAILED DESCRIPTION

Our methods are described below in detail. The microorganism used is a microorganism that can produce alcohol as a major product from a material containing pentose, and has an enhanced Entner-Doudoroff (ED) pathway.

The “material containing pentose” means a material containing as a carbon source a hexose such as glucose, mannose, galactose or fructose; a disaccharide such as sucrose, lactose, maltose, trehalose or cellobiose, or a sugar such as glycerol, in addition to a pentose such as xylose, arabinose, ribulose, ribose or xylulose. However, the “material containing pentose” is not limited to these.

The term “as a major product” means that the microorganism produces alcohol at not less than 20%.

Examples of the “alcohol” include ethanol; butanol; isobutanol; butanediols such as 1,2-butanediol, 1,3-butanediol, 1,4-butanediol, and 2,3-butanediol; methanol; sugar alcohols such as erythritol, sorbitol, and xylitol; 1,3-propanediol; and glycerol. The “alcohol” is preferably ethanol, isobutanol, butanol, or xylitol, more preferably ethanol.

The term “alcohol can be produced from a material containing pentose” means a capacity to assimilate and metabolize pentose present in a medium when the microorganism is cultured therein. Specific examples of the capacity to assimilate pentose include, but are not limited to, the capacity to assimilate a pentose(s) such as xylose, arabinose, ribulose, ribose, and/or xylulose. The capacity to assimilate pentose may be an inherent property of a microorganism. Alternatively, the capacity may be a property given to or enhanced in a microorganism which does not originally have the capacity to assimilate pentose, by breeding.

Examples of the technique for the breeding used herein include mutagenesis, cell fusion, and genetic recombination. Examples of the technique of mutagenesis include chemical treatment and ultraviolet treatment.

When the pentose-assimilating performance is given and/or enhanced by breeding, a gene fragment encoding an enzyme related to assimilation of pentose, for example, the following enzyme(s), may be introduced into a microorganism, or an expression regulatory sequence such as a gene promoter may be replaced with a stronger sequence. Examples of candidates of the enzyme related to assimilation of pentose include xylose isomerase (XI), xylose reductase (XR), and xylose dehydrogenase (XDH).

Examples of the microorganism inherently having a pentose-assimilating capacity include Escherichia coli, Bacillus subtilis, Bacillus palidus, Bacillus stearothermophilus, Salmonella typhimurium, Mycobacterium smegmatis, Azospirillum brasiliense, Herbaspirillum seropedicae, Bifidobacterium longum, Trichoderma reesei, Lactobacillus plantarum, Ambrosiozyma monospora, Burkholderia uboniae, Pichia Guilliemondii, Scheffersomyces stipitis (Pichia stipitis), Candida arabinofermentans, Candida intermedia, Candida tropicalis, Candida parapsilosis, Kluyveromyces maxianus, Brettanomyces bruxellensis, and Bretannomyces naardenensis. Examples of the technique for the breeding include mutagenesis, cell fusion, and genetic recombination. Examples of the technique of mutagenesis include chemical treatment and ultraviolet treatment.

The “microorganism having an enhanced Entner-Doudoroff (ED) pathway” may be a microorganism prepared by enhancement of the ED pathway by breeding of a microorganism originally having genes related to the ED pathway as a property of the microorganism, or may be a microorganism prepared by giving and/or enhancing the ED pathway by breeding of a microorganism which does not originally have the ED pathway.

The genes related to the ED pathway are genes encoding 6-phosphogluconate dehydratase (hereinafter referred to as EDD), 2-keto-3-deoxy-6-phosphogluconate aldolase (hereinafter referred to as EDA), glucose-6-phosphate dehydrogenase (hereinafter referred to as “ZWF” for short), and 6-phosphogluconolactonase (hereinafter referred to as PGL).

The microorganism having an enhanced ED pathway means a microorganism whose activity/activities of EDD and/or EDA and/or ZWF and/or PGL is/are higher than the activity/activities before the enhancement of the ED pathway. Examples of such cases include when the number of EDD or EDA or ZWF or PGL molecules per cell has increased, and when the specific activity of EDD or EDA or ZWF or PGL per EDD or EDA or ZWF or PGL molecule has increased. Examples of the method of measuring the activity of the ED pathway include determination of the activity by measurement of the activity of the reaction in which pyruvic acid is produced from 6-phosphogluconate through 2-keto-3-oxy-6-phosphogluconate. Detection of the reaction can be carried out by reacting a microbial cell homogenate with 6-phosphogluconate, and converting the produced pyruvic acid to lactic acid using lactate dehydrogenase, while measuring the amount of decrease in NADH.

The method of enhancing the EDD activity and/or EDA activity and/or ZWF activity and/or PGL activity of the microorganism is not limited. For example, a gene fragment encoding EDD and/or EDA and/or ZWF and/or PGL may be linked to a vector, preferably a multi-copy type vector, which functions in the microorganism of interest, to prepare a recombinant DNA, and the resulting vector may be introduced to this microorganism. When all of the EDD activity and the EDA activity and the ZWF activity and the PGL activity are to be enhanced, the gene fragments encoding the EDD and EDA and ZWF and PGL may be separately loaded on different vectors, but the gene fragments are preferably loaded on the same vector.

The genes encoding the enzymes EDD and EDA and ZWF and PGL constituting the ED pathway, that is, edd and eda and zwf and pgl of the genus Zymomonas, respectively, may be any of these genes derived from microorganisms having the ED pathway. More specifically, those genes have been cloned from Escherichia coli, Zymomonas mobilis, and the like. The genes edd and eda and zwf and pgl can be obtained by PCR using primers prepared based on sequences of these genes (PCR: White, T. J. et al., Trends Genet. 5, 185 (1989)), or by hybridization using probes prepared based on sequences of the genes. For example, an operon fragment containing edd and eda and zwf and pgl can be obtained by the later-described PCR method. In a similar manner, edd and eda and zwf and pgl from other microorganisms can also be obtained. Examples of the conditions for the hybridization include those in which washing is carried out at a salt concentration corresponding to 1×SSC and 0.1% SDS at a temperature of 60° C.

Chromosomal DNA can be prepared from the microorganism which is a DNA donor by, for example, using “Dr. GenTLE” (manufactured by Takara Bio Inc.).

The edd and/or eda and/or zwf and/or pgl gene(s) amplified by the PCR may be linked to a vector DNA capable of autonomous replication in cells such as Escherichia coli to prepare a recombinant DNA, and the recombinant DNA may then be introduced to Escherichia coli to make the subsequent operations simple. Examples of the vector that is capable of autonomous replication in cells of Escherichia coli include pMW219, pSTV28, pUC18, pUC19, pHSG299, pHSG399, pHSG398, RSF1010, pBR322, and pACYC184. Examples of the selection marker for the E. coli include antibiotic resistance genes such as the ampicillin resistance gene and the kanamycin resistance gene.

Examples of the regulatory sequence include a GAPDH (glyceraldehyde-3-phosphate dehydrogenase) promoter, ADH (alcohol dehydrogenase) promoter, and GAPDH terminator. However, the expression vector is not limited to these. By introducing the edd and/or eda and/or zwf and/or pgl gene(s) derived from Zymomonas or the like downstream of the promoter of the expression vector, a vector capable of expressing the gene(s) can be obtained.

For introduction of the ED pathway gene expression vector or the PCR fragment obtained as described above to the microorganism, a method such as transformation, transduction, transfection, co-transfection, or electroporation may be used.

An increased copy number(s) of the edd and/or eda and/or zwf and/or pgl gene(s) can be obtained by allowing a large number of copies of the gene(s) to be present on the chromosomal DNA. Introduction of the large number of copies of the edd and/or eda and/or zwf and/or pgl gene(s) into the chromosomal DNA of the microorganism is carried out by homologous recombination using, as targets, sequences that are present in a large number of copies on the chromosomal DNA. Alternatively, as disclosed in JP 2-109985 A, the edd and/or eda and/or zwf and/or pgl gene(s) may be loaded on a transposon, and transposition of the transposon may be allowed to occur to achieve introduction of the genes in a large number of copies into the chromosomal DNA.

Enhancement of the activity/activities of EDD and/or EDA and/or ZWF and/or PGL can be achieved not only by the above-described methods based on the genes, but also by replacement of an expression regulatory sequence(s) such as the promoter(s) for the edd and/or eda and/or zwf and/or pgl gene(s) on the chromosomal DNA or on a plasmid, with a stronger sequence(s). Known examples of strong promoters include the lac promoter and the trc promoter. Modification of the expression regulatory sequence(s) may be carried out in combination with the increasing of the copy number(s) of the edd and/or eda and/or zwf and/or pgl gene(s).

The capacity of the microorganism to produce alcohol may be an inherent property of the microorganism. Alternatively, the capacity may be a property given to or enhanced in the microorganism by breeding.

When the alcohol production capacity is to be given and/or enhanced by breeding, a gene fragment encoding an enzyme that catalyzes biosynthesis of alcohol, for example, the following enzyme(s), may be introduced to a microorganism, or an expression regulatory sequence such as a gene promoter may be replaced with a stronger sequence.

When the alcohol of interest is ethanol, examples of the enzyme include alcohol dehydrogenase, phosphoenol pyruvate carboxylase, pyruvate decarboxylase (pdc), pyruvate kinase (pyk), enolase (eno), phosphoglyceromutase (gpmA), phosphoglycerate kinase (pgk), glyceraldehyde-3-phosphate dehydrogenase (gap), triose phosphate isomerase (tpi), fructose bisphosphate aldolase (fba), phosphofructokinase (pfk), and glucose phosphate isomerase (pgi). When the alcohol of interest is butanol or isobutanol, examples of the enzyme include acetolactate synthase, ketol acid reductoisomerase, dihydroxy acid dehydratase, 2-keto acid decarboxylase, alcohol dehydrogenase, ketol acid reductoisomerase, acetohydroxy acid dehydratase, valine dehydrogenase or transaminase, valine carboxylase, omega transaminase, and branched chain amino acid alcohol dehydrogenase.

Other examples of the method include a method in which the activity of an enzyme that catalyzes a reaction to produce another compound in a pathway branched from the biosynthetic pathway for the alcohol of interest is decreased or removed to give and/or enhance the alcohol productivity.

The microorganism is not limited as long as it is a microorganism having an enhanced ED pathway. Examples of the microorganism include the genus Escherichia. The microorganism is preferably Escherichia coli. More specifically, the KO11 strain is preferred.

By culturing the microorganism having an enhanced ED pathway, alcohol can be produced in the medium with a yield of at least not less than 20% based on sugar consumption. It is advantageous to produce alcohol in the medium with a yield of at least not less than 20% based on sugar consumption also from the viewpoint of the energy efficiency because, for example, the distillation yield of the alcohol can be increased in a later step. The alcohol can be produced in the medium more preferably with a yield of not less than 30%, still more preferably with a yield of not less than 40%.

When alcohol is produced from a material containing pentose using the microorganism having an enhanced ED pathway, the production of the alcohol from the material containing pentose can be achieved with an increased yield based on sugar consumption compared to before the enhancement of the ED pathway. The extent of the increase is preferably not less than 5% compared to before the enhancement of the ED pathway. The yield based on sugar consumption (%) can be calculated according to Equation (1).

Yield based on sugar consumption (g/g)={the concentration of alcohol in the medium after the culture (g/L)×the amount of the medium (L)}÷{the sugar concentration in the medium before the culture (g/L)×the amount of the medium−the concentration of the remaining sugar in the medium after the culture (g)×the amount of the medium (L)}×100 (1).

The medium used in the method of producing alcohol is not limited as long as it promotes the growth of the microorganism to be cultured, to thereby allow favorable production of the alcohol of interest. The medium is preferably a liquid medium containing a carbon source, nitrogen source, inorganic salts, and, if necessary, organic micronutrients such as amino acids and vitamins. Examples of the carbon source include those described above, as well as saccharified starch solutions containing these sugars; sweet potato molasses; sugar beet molasses; high test molasses; and further, organic acids such as acetic acid; alcohols such as ethanol; and glycerin. These may be used either individually or in combination with other carbon sources. Among these, glucose, xylose, and arabinose are preferred. Examples of the nitrogen source include ammonia gas, aqueous ammonia, ammonium salts, urea, nitric acid salts, and other organic nitrogen sources used supplementarily. Examples of the organic nitrogen sources include oil cakes, soybean hydrolysates, casein digests, amino acids, vitamins, corn steep liquors, yeasts, yeast extracts, meat extracts, peptides such as peptone, and cells of various fermentation microorganisms and hydrolysates thereof. Examples of the inorganic salts which may be added as appropriate include phosphoric acid salts, magnesium salts, calcium salts, iron salts, and manganese salts. When the microorganism requires a particular nutrient for its growth, the nutrient may be added as a preparation or a natural product containing it. An anti-forming agent may also be used, if necessary.

When the expression vector introduced is to be retained in the microorganism, it is preferred to use a medium suitable for application of a selection pressure for a selection marker. Examples of the medium include synthetic media prepared by removing an amino acid that matches the selection marker retained in the vector.

The method of culturing the microorganism in the method of producing alcohol is not limited. The alcohol can be produced by, for example, the following culture method. First, the microorganism is precultured, and the preculture liquid is then transferred into a fresh medium to perform main culture. By this, the alcohol can be produced in the culture liquid. The culture temperature is not limited as long as the growth of the microbial strain is not substantially inhibited, and as long as the alcohol of the interest can be produced. The culture temperature is preferably a temperature of 20 to 40° C. As the culture method, any of static culture, spinner culture, and shake culture may be employed. The growth of the microbial cells proceeds either under aerobic conditions or anaerobic conditions. The reaction may be carried out by any of a continuous process, fed-batch process, and batch process. At an appropriate time after the beginning of the culture, the medium may be collected, and the alcohol may be separated and purified. The method of separation and purification is not limited. When the alcohol is ethanol, butanol, isobutanol, 2,3-butanediol, methanol or the like, examples of the method include distillation and use of an osmotic evaporation membrane. When the alcohol is erythritol, sorbitol, xylitol or the like, examples of the method include ammonium sulfate precipitation, removal of protein by zinc hydroxide adsorption, decoloration by activated carbon adsorption, and desalting with an ion-exchange resin.

The method of measuring the obtained alcohol is not limited, and examples of the method include methods using HPLC and methods using “F-kit” (manufactured by Roche).

### EXAMPLES

Our methods are described below by way of Examples. However, this disclosure is not limited by the Examples. The yield based on sugar consumption was calculated according to Equation (1).

### Reference Example 1 Construction of pRA17, Plasmid for Strong Expression of ED Pathway-Related Genes

Genes related to the ED pathway zwf, edd, eda, and pgl were cloned from Zymomonas mobilis subsp. mobilis ZM4. From Zymomonas mobilis subsp. mobilis ZM4, genomic DNA was extracted using “Dr. GenTLE” (manufactured by Takara Bio Inc.), and the genes zwf-edd, eda, and pgl were amplified by PCR (which was carried out using “KOD Plus” manufactured by Takara Bio Inc.) using the extracted genomic DNA as a template. In Zymomonas mobilis subsp. mobilis ZM4, zwf and edd form an operon. In view of this, primers that can amplify these genes at once, ED005 (SEQ ID NO:5) and ED006 (SEQ ID NO:6) were designed, and a DNA fragment containing both genes was amplified by PCR. Similarly, as primers for amplification of eda, ED007 (SEQ ID NO:7) and ED008 (SEQ ID NO:8) were designed, and, as primers for amplification of pgl, ED009 (SEQ ID NO:9) and ED010 (SEQ ID NO:10) were designed. Using these primers, a gene fragment was amplified for each of the genes.

From Escherichia coli JM109, genomic DNA was extracted using “Dr. GenTLE”, and gene fragments each containing a GAPDH promoter were amplified by PCR using the extracted genomic DNA as a template, and using each of two pairs of primers. The gene fragment amplified using ED001 (SEQ ID NO:1) and ED002 (SEQ ID NO:2) was provided as the GAPDH promoter fragment 1, and the gene fragment obtained using ED003 (SEQ ID NO:3) and ED004 (SEQ ID NO:4) was provided as the GAPDH promoter fragment 2.

Using the GAPDH promoter fragment 1 and the above-described zwf-edd as templates, and using the primers ED001 and ED006, GAPDH promoter-zwf-edd was amplified. Using the GAPDH promoter fragment 2 and eda as templates, and using the primers ED003 and ED008, GAPDH promoter-eda was amplified. By using GAPDH promoter-eda and pgl as templates, and using ED003 and ED010 as primers, GAPDH promoter-eda-pgl was amplified to prepare a GAPDH promoter-zwf-edd fragment, followed by digesting it with SacI and XbaI, and inserting the resulting fragment into the multicloning site of pUC18, to prepare pRA15. The GAPDH promoter-eda-pgl fragment was treated with XbaI and SalI, and the resulting fragment was similarly inserted into the multicloning site of pUC 18, to prepare pRA16. pRA15 was treated with SacI and XbaI, and the GAPDH promoter-zwf-edd fragment obtained by the cleavage was inserted into pRA16, to prepare pRA17.

### Reference Example 2 Preparation of E. coli Having Enhanced ED Pathway

The ethanol-fermenting E. coli KO11 was transformed with the pRA17 prepared, to provide the E. coli RA34 strain having an enhanced ED pathway. To provide a microorganism before enhancement of the ED pathway of the RA34 strain, KO11 was transformed with pUC18 to prepare the RA36 strain, and the prepared E. coli, whose ED pathway is not enhanced, was used in Comparative Examples.

### Reference Example 3 Measurement of Activity of ED Pathway

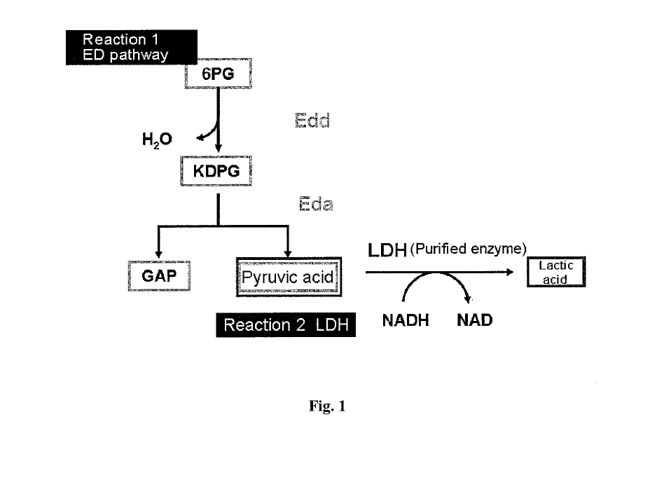

To test enhancement of the activity of the ED pathway of the E. coli whose ED pathway is enhanced, activity measurement was carried out using bacterial cell homogenates of the RA36 strain and the RA34 strain prepared in Reference Example 2. The reaction pathway used for the measurement of the ED pathway activity is shown in FIG. 1. The measurement of the activity of the ED pathway was carried out by reacting 6-phosphogluconate with the bacterial cell homogenates of the RA36 strain and the RA34 strain, and utilizing the reaction in which pyruvic acid is produced from 6-phosphogluconate through 2-keto-3-oxy-6-phosphogluconate. Based on the amount of decrease in NADH upon conversion of the produced pyruvic acid to L-lactic acid by reaction with L-lactate dehydrogenase and NADH, the amount of the produced pyruvic acid was measured for indirect observation of the activity of the ED pathway.

The method of measuring the activity of the ED pathway is described below. For preculture, a loopful of bacterial cells of each of the RA36 strain and the RA34 strain were inoculated into 5 mL of LB medium contained in a test tube. The cells were cultured at 37° C. for 16 hours with shaking (125 rpm).

For main culturing, 500 μL (1 vol %) of the preculture liquid was inoculated into 50 mL of LB medium contained in a 500-mL Erlenmeyer flask. The cells were then cultured at 37° C. for 6 hours with shaking on a rotary shaker (125 rpm).

For pretreatment, 1 mL of the main culture liquid was collected, and the cells were precipitated using a centrifuge. After discarding the supernatant, the cells were resuspended in M9 salt. Subsequently, the bacterial cells were precipitated again using a centrifuge, and washed twice with M9 salt, followed by resuspension in 1 mL of Bis-Tris buffer (manufactured by Dojindo Laboratories). Into a tube for bead beating containing about 100 μL of zirconia beads, the cell suspension was added, and bead beating at 4000 rpm for 1 minute was carried out five times (at 3-minute intervals). Centrifugation was carried out at 10,000×g for 10 minutes at 4° C., and the resulting supernatant was transferred into another tube.

For activity measurement, an ED pathway reaction solution was prepared as follows. With 140 μL of the bacterial homogenate, an MgCl₂ solution (final concentration, 10 mM) and a 6-polypropylene glycol solution (final concentration, 2 mM) were mixed to provide 200 μL of an ED pathway reaction solution. The resulting ED pathway reaction solution was incubated at 30° C. for 30 minutes. The remaining homogenate was used for measurement of the amount of protein. For protein measurement, Quick Start Bradford protein assay (manufactured by BIORAD) was used. The amount of each solution for the LDH reaction was adjusted for 100 μL of the ED pathway reaction solution using Bis-Tris buffer (manufactured by Dojindo Laboratories) as follows: an NADH solution (final concentration, 1 mM) and LDH (final concentration, 0.63 U/mL).

The Bis-Tris buffer was preliminarily incubated at 30° C., and NADH and the ED pathway reaction solution were mixed therewith immediately before the reaction. After adding the resulting mixture to a cuvette containing L-lactate dehydrogenase solution (derived from Leuconostoc meseuteroids, manufactured by Oriental Yeast Co., Ltd.), the decrease in the absorbance at 340 nm due to the decrease in NADH was monitored for 10 minutes from the beginning of the reaction over time. Since the amount of decrease in NADH in this reaction is equal to the amount of pyruvic acid produced, the ED pathway activity can be obtained. The activity was standardized to the amount of protein. The RA34 strain was also subjected to the measurement in the same manner. While no activity of the ED pathway could be detected for the RA36 strain, the activity of the ED pathway could be detected for RA34, so that the RA34 strain was confirmed to have an enhanced ED pathway.

### Reference Example 4 Method of Quantifying Products in Medium

The following are conditions for measurement of the concentrations of components in the culture liquid by high-performance liquid chromatography (HPLC, manufactured by Shimadzu Corporation).

The concentrations of xylose, arabinose, and ethanol were quantified under the HPLC conditions described below based on comparison with standard samples.

Column: Shodex SH1011 (manufactured by Showa Denko K. K.)

Mobile phase: 5 mM sulfuric acid (flow rate, 0.6 mL/minute)

Reaction liquid: none

Detection method: RI (differential refractive index)

Temperature: 65° C.

Lactic acid, acetic acid, and formic acid in the culture liquid were quantified under the HPLC conditions described below based on comparison with standard samples.

Column: Shim-Pack SPR-H (manufactured by Shimadzu Corporation)

Mobile phase: 5 mMp-toluenesulfonic acid (flow rate, 0.8 mL/min.)

Reaction solution: 5 mM p-toluenesulfonic acid, 20 mM Bis-Tris, 0.1 mM EDTA.2Na (flow rate: 0.8 mL/min.)

Detection method: Electric conductivity

Temperature: 45° C.

### Example 1 Production of Ethanol Under Aerobic Conditions Using Xylose as Material by E. coli Having Enhanced ED Pathway

To study the influence of the enhancement of the ED pathway on production of ethanol using pentose as a material, the ethanol productivity of the RA34 strain prepared in Reference Example 2 was evaluated by culture. As an evaluation medium, a medium containing 20 g/L xylose, 20 g/L (NH₄)₂SO₄, 1 g/L NH₄Cl, 0.4 g/L MgSO₄.7H₂O, 0.525 g/L NaCl, 3 g/L KH₂PO₄, 6 g/L Na₂HPO₄.12H₂O, 7.35 mg/L CaCl₂.2H₂O, 20 mg/L FeSO₄.7H₂O, 2 mg/L MnSO₄.4H₂O, 0.02 mg/L ZnSO₄.7H₂O, 0.54 mg/L CuSO₄.5H₂O, 0.08 mg/L (NH₄)₆Mo₇O₂₄.4H₂O, 0.176 mg/L Na₂MoO₄2H₂O, 0.176 mg/L Na₂B₄O₇.7H₂O, 1.74 mg/L FeCl₃.6H₂O, 0.0144 mg/L MnCl₂.4H₂O, 0.03 mg/L Biotine, and 1 mg/L Thiamine.HCl was prepared. The pH of the medium was kept around 6.0 using citrate-phosphate buffer. The culture was carried out with stirring at 120 rpm.

For preculturing, a loopful of bacterial cells of the RA34 strain were inoculated into 5 mL of LB medium contained in a test tube. The cells were cultured at 37° C. for 16 hours with reciprocal shaking at 125 rpm.

For main culturing, 20 mL of the evaluation medium described above was placed in a 50-mL Erlenmeyer flask, and 500 μL of the preculture liquid was inoculated to the medium, followed by performing culture. The xylose concentration and the ethanol concentration in the medium after 24 hours of the culture were measured by the method described in Reference Example 4. The concentration of the remaining sugar, the yield of ethanol based on sugar consumption, and the ethanol production rate are shown in Table 1.

TABLE 1

| | | | Concentration of | Concentration of | Ethanol yield | |
|---|---|---|---|---|---|---|
| | | | remaining sugar | ethanol in | produced | Ethanol |
| | Culture | | in medium after | medium after | based on sugar | production |
| | conditions | Sugar source | culture (g/L) | culture (g/L) | consumption (%) | rate (g/L/h) |
| Example 1 | Aerobic | Xylose | 0 | 8 | 38.5 | 0.33 |
| Comparative | Aerobic | Xylose | 8.7 | 3.2 | 26.2 | 0.13 |
| Example 1 | | | | | | |
| Example 2 | Anaerobic | Xylose | 11.2 | 4.5 | 47.4 | 0.19 |
| Comparative | Anaerobic | Xylose | 13.2 | 3.1 | 42 | 0.13 |
| Example 2 | | | | | | |
| Example 3 | Aerobic | Arabinose | 0 | 7.2 | 38 | 0.3 |
| Comparative | Aerobic | Arabinose | 6.2 | 3.9 | 30.2 | 0.16 |
| Example 3 | | | | | | |
| Example 4 | Anaerobic | Arabinose | 10.1 | 4.3 | 47.6 | 0.18 |
| Comparative | Anaerobic | Arabinose | 13.4 | 2.4 | 39.9 | 0.1 |
| Example 4 | | | | | | |

### Comparative Example 1 Production of Ethanol Under Aerobic Conditions Using Xylose as Material by E. coli Whose ED Pathway is not Enhanced

Evaluation by culture was carried out under the same conditions as in Example 1 except that the RA36 strain prepared in Reference Example 2 was used instead of the RA34 strain. The results after 24 hours of the culture are shown in Table 1. As a result, it was shown that production of ethanol by E. coli whose ED pathway is not enhanced results in a lower consumption of xylose, which is a pentose, as well as a lower productivity of ethanol, compared to those in Example 1.

### Example 2 Production of Ethanol Under Anaerobic Conditions Using Xylose as Material by E. coli Having Enhanced ED Pathway

For preculturing, a loopful of bacterial cells of the RA34 strain were inoculated into 5 mL of LB medium contained in a test tube. The cells were cultured at 37° C. for 16 hours with reciprocal shaking at 125 rpm. For main culturing, 50 mL of the evaluation medium described above was placed in a 50-mL Erlenmeyer flask at a xylose concentration of 20 g/L, and 500 μL of the preculture liquid was inoculated to the medium, followed by performing culture. During the culturing, anaerobic conditions were maintained using a check valve. The xylose concentration and the ethanol concentration in the culture liquid after 24 hours of the culture were measured by the method described in Reference Example 4. The concentration of the remaining sugar, the yield of ethanol based on sugar consumption, and the ethanol production rate are shown in Table 1.

### Comparative Example 2 Production of Ethanol Under Anaerobic Conditions Using Xylose as Material by E. coli Whose ED Pathway is not Enhanced

Evaluation by culture was carried out under the same conditions as in Example 2 except that the RA36 strain prepared in Reference Example 2 was used instead of the RA34 strain. The results after 24 hours of the culture are shown in Table 1. As a result, it was shown that production of ethanol by E. coli whose ED pathway is not enhanced results in a lower consumption of xylose, which is a pentose, as well as a lower productivity of ethanol, compared to those in Example 2.

### Example 3 Production of Ethanol Under Aerobic Conditions Using Arabinose as Material by E. coli Having Enhanced ED Pathway

Culturing was carried out under the same conditions as in Example 1 except that the evaluation medium was prepared such that 20 g/L arabinose is contained instead of 20 g/L xylose. The arabinose concentration in the culture liquid after 24 hours of the culture was measured by the method described in Reference Example 4. The arabinose concentration and the ethanol concentration in the culture liquid after 24 hours of the culture were measured by the method described in Reference Example 4. The concentration of the remaining sugar, the yield of ethanol based on sugar consumption, and the ethanol production rate are shown in Table 1.

### Comparative Example 3 Production of Ethanol Under Aerobic Conditions Using Arabinose as Material by E. coli Whose ED Pathway is not Enhanced

Evaluation by culture was carried out under the same conditions as in Example 3 except that the RA36 strain prepared in Reference Example 2 was used instead of the RA34 strain. The results after 24 hours of the culture are shown in Table 1. As a result, it was shown that production of ethanol by E. coli whose ED pathway is not enhanced results in a lower consumption of arabinose, which is a pentose, as well as a lower productivity of ethanol, compared to those in Example 3.

### Example 4 Production of Ethanol Under Anaerobic Conditions Using Arabinose as Material by E. coli Having Enhanced ED Pathway

Culturing was carried out under the same conditions as in Example 2 except that the evaluation medium was prepared such that 20 g/L arabinose is contained instead of 20 g/L xylose. The arabinose concentration and the ethanol concentration in the culture liquid after 24 hours of the culture were measured by the method described in Reference Example 4. The concentration of the remaining sugar, the yield of ethanol based on sugar consumption, and the ethanol production rate are shown in Table 1.

### Comparative Example 4 Production of Ethanol Under Anaerobic Conditions Using Arabinose as Material by E. coli Whose ED Pathway is not Enhanced

Evaluation by culture was carried out under the same conditions as in Example 4 except that the RA36 strain before the enhancement of the ED pathway prepared in Reference Example 2 was used instead of the RA34 strain. The results after 24 hours of the culture are shown in Table 1. As a result, it was shown that production of ethanol by E. coli whose ED pathway is not enhanced results in a lower consumption of arabinose, which is a pentose, as well as a lower productivity of ethanol, compared to those in Example 4.

### Example 5 By-Products Produced in Ethanol Fermentation by E. coli Having Enhanced ED Pathway

Acetic acid, lactic acid, and formic acid in the culture liquids obtained in Examples 1 to 4 were measured by the method described in Reference Example 4. Table 2 shows the yields of acetic acid, lactic acid, and formic acid based on sugar consumption.

### Comparative Example 5 By-Products Produced in Ethanol Fermentation by E. coli Whose ED Pathway is not Enhanced

Acetic acid, lactic acid, and formic acid in the culture liquids obtained in Comparative Examples 1 to 4 were measured by the method described in Reference Example 4. Table 2 shows the yields of acetic acid, lactic acid, and formic acid based on sugar consumption. As a result, it was shown that production of ethanol by E. coli whose ED pathway is not enhanced results in increased production of products other than ethanol, as by-products.

From the results of Examples 1 to 5 and Comparative Examples 1 to 5, we found that, by producing alcohol from a material containing pentose using a microorganism having an enhanced ED pathway, the yield based on sugar consumption can be increased by not less than 5% as compared to the yield before the enhancement of the ED pathway.

TABLE 2

| | | | Acetic | Lactic | Formic |
|---|---|---|---|---|---|
| | Culture | Sugar | acid | acid | acid |
| | conditions | source | (g/L) | (g/L) | (g/L) |
| Example 1 | Aerobic | Xylose | 3.2 | 0 | 0.021 |
| Comparative | Aerobic | Xylose | 10.0 | 0.1 | 0.178 |
| Example 1 | | | | | |
| Example 2 | Anaerobic | Xylose | 13.0 | 0 | 0.095 |
| Comparative | Anaerobic | Xylose | 15.0 | 0 | 0.294 |
| Example 2 | | | | | |
| Example 3 | Aerobic | Arabinose | 4.2 | 0 | 0 |
| Comparative | Aerobic | Arabinose | 7.6 | 0 | 0.015 |
| Example 3 | | | | | |
| Example 4 | Anaerobic | Arabinose | 12.0 | 0.2 | 0.131 |
| Comparative | Anaerobic | Arabinose | 17.0 | 0.2 | 0.336 |
| Example 4 | | | | | |

### Example 6 Distillation of Ethanol Produced from Culture Liquids of E. coli Having Enhanced ED Pathway Using Pentose as Material

The concentration of ethanol after the fermentation step is only several percent. To increase the concentration so that the ethanol can be used as a fuel, distillation of the culture liquids obtained in Examples 1 to 4 was carried out. The operation was carried out according to a method disclosed in a Patent Document (JP 2008-182925 A) and the like. The results are shown in Table 3.

### Comparative Example 6 Distillation of Ethanol Produced from Culture Liquids of E. coli Whose ED Pathway is not Enhanced Using Pentose as Material

Under the same conditions as in Example 6, distillation of the culture liquids obtained in Comparative Examples 1 to 4 was carried out by the same method as in Example 6. The results are shown in Table 3. It was shown, as a result, that the distillation yield of ethanol is low since, as shown in Example 5 and Comparative Example 5, production of ethanol by E. coli whose ED pathway is not enhanced results in production of a large amount of by-products in the culture liquid.

TABLE 3

| | | | Ethanol |
|---|---|---|---|
| | Culture | Sugar | distillation |
| | conditions | source | yield (%) |
| Example 1 | Aerobic | Xylose | 97 |
| Comparative | Aerobic | Xylose | 90 |
| Example 1 | | | |
| Example 2 | Anaerobic | Xylose | 96 |
| Comparative | Anaerobic | Xylose | 92 |
| Example 2 | | | |
| Example 3 | Aerobic | Arabinose | 97 |
| Comparative | Aerobic | Arabinose | 90 |
| Example 3 | | | |
| Example 4 | Anaerobic | Arabinose | 96 |
| Comparative | Anaerobic | Arabinose | 91 |
| Example 4 | | | |

### INDUSTRIAL APPLICABILITY

Our methods can be used for a method of production of alcohol from a material containing pentose.

1.-7. (canceled)

8. A method of producing alcohol, comprising culturing a microorganism capable of producing alcohol as a major product from a material containing pentose, wherein said microorganism is a microorganism having an enhanced Entner-Doudoroff (ED) pathway, and alcohol yield from xylose or arabinose based on sugar consumption is improved compared to alcohol yield based on sugar consumption before enhancement of the ED pathway.

9. The method according to claim 8, wherein the alcohol yield from xylose or arabinose based on sugar consumption in said microorganism is improved by not less than 5% compared to the alcohol yield from xylose or arabinose based on sugar consumption before the enhancement of the ED pathway.

10. The method according to claim 8, wherein said microorganism is a microorganism exhibiting enhanced expression of a gene(s) encoding at least one selected from the group consisting of glucose-6-phosphate dehydrogenase, 6-phosphogluconolactonase, phosphogluconate dehydratase, and 2-dehydro-3-deoxy-6-phosphogluconate aldolase.

11. The method according to claim 8, wherein the alcohol is ethanol.

12. The method according to claim 8, wherein said microorganism is a microorganism belonging to the genus Escherichia.

13. The method according to claim 8, wherein said microorganism is Escherichia coli.

14. The method according to claim 8, wherein said pentose is xylose or arabinose.

15. The method according to claim 9, wherein said microorganism is a microorganism exhibiting enhanced expression of a gene(s) encoding at least one selected from the group consisting of glucose-6-phosphate dehydrogenase, 6-phosphogluconolactonase, phosphogluconate dehydratase, and 2-dehydro-3-deoxy-6-phosphogluconate aldolase.

16. The method according to claim 9, wherein the alcohol is ethanol.

17. The method according to claim 10, wherein the alcohol is ethanol.

18. The method according to claim 9, wherein said microorganism is a microorganism belonging to the genus Escherichia.

19. The method according to claim 10, wherein said microorganism is a microorganism belonging to the genus Escherichia.

20. The method according to claim 11, wherein said microorganism is a microorganism belonging to the genus Escherichia.

21. The method according to claim 9, wherein said microorganism is Escherichia coli.

22. The method according to claim 10, wherein said microorganism is Escherichia coli.

23. The method according to claim 11, wherein said microorganism is Escherichia coli.

24. The method according to claim 12, wherein said microorganism is Escherichia coli.

25. The method according to claim 9, wherein said pentose is xylose or arabinose.

26. The method according to claim 10, wherein said pentose is xylose or arabinose.

27. The method according to claim 11, wherein said pentose is xylose or arabinose.
